(19) United States
(12) Patent Application Publication
(10) Pub. No.: US 2013/0330799 A1
(43) Pub. Date: Dec. 12, 2013

(54) CRUDE OIL DEGRADING MICROORGANISMS AND METHODS FOR THEIR ENHANCEMENT

(71) Applicant: Dmitriy Ovcharenko, Austin, TX (US)

(72) Inventor: Dmitriy Ovcharenko, Austin, TX (US)

(73) Assignee: ALTOGEN LLC, Austin, TX (US)

(21) Appl. No.: 13/490,508

(22) Filed: Jun. 7, 2012

(51) Int. Cl. C12N 1/20; C12N 11/00; A62D 3/02; C12N 1/26

(52) U.S. Cl. 435/174; 435/248; 435/252.1; 435/262.5

(57) ABSTRACT

Certain embodiments are directed to methods of enhancing the petroleum degrading abilities of bacteria and the resulting bacterial composition. In certain aspects the bacteria are selected for degradation of a petroleum having a particular composition profile and/or for environmental robustness.

### STATEMENT REGARDING FEDERALLY FUNDED RESEARCH

none

### REFERENCE TO SEQUENCE LISTING

A sequence listing required by 37 CFR 1.821-1.825 is being submitted electronically with this application. The sequence listing is incorporated herein by reference.

### BACKGROUND

The present invention relates to bioremediation. In certain embodiments, the invention relates to crude oil-degrading microorganisms and methods for enrichment and isolation of crude oil-degrading microorganisms.

Crude oil refers to a mixture of compounds that can be categorized into four fractions: (1) saturates (or alkanes); (2) aromatics, including for example benzene, toluene, ethylbenzene and xylenes, and polyaromatic hydrocarbons (PAHs); (3) resins, which are compounds containing nitrogen, sulphur, and oxygen, that are dissolved in oil; and (4) asphaltenes, which are large and complex molecules that are colloidally dispersed in oil. The relative proportions of these fractions are dependent on many factors, including for example source, age, and migration. Of these fractions, the shorter alkane chain compounds and the lighter aromatics tend to be more readily biodegradable.

Biodegradation refers to the chemical breakdown or transformation of substances through biochemical reactions, such as those carried out by microorganisms. Bioremediation refers to the use of a biological process to degrade chemical contaminants in the environment, for example crude oil that may be released after an oil spill. Bioremediation has shown promise as a supplemental treatment option for oil spill cleanup. Bioremediation agents include microbiological cultures, enzyme additives, and nutrient additives that significantly increase the rate of biodegradation to mitigate the effects of oil discharge. The addition of known oil-degrading bacteria to a contaminated site to effect or enhance biodegradation of crude oil and its components has been referred to as bioaugmentation.

Hydrocarbon-degrading bacteria can assimilate and metabolize hydrocarbons that are present in petroleum. Some oil-degrading bacteria are naturally occurring in soil and water environments. Rhodococcus spp. are the most abundant alkane-degrading bacteria, in many natural environments, and Pseudomonas spp. and Alcanivorax borkumensis may become enriched following contamination events such as crude oil spills. Upon contamination with crude oil, the concentrations of these bacteria may increase. However, the natural bioremediation process is frequently inefficient, because the bacteria require nutrients and oxygen and are often effective only at the edge of the oil spill. Also, indigenous microbial populations may not be capable of degrading the wide range of chemical compounds such as those present in complex hydrocarbon mixtures.

Recently, considerable efforts have focused on isolating microorganisms that can degrade polycyclic aromatic hydrocarbons (PAHs), with the goal of understanding the fate of these toxic compounds after they are deposited in natural environments. The EPA has listed PAHs as priority pollutants in ecosystems since the 1970s. Although many PAHs are recalcitrant to biodegradation, numerous bacteria are known to catabolize PAHs as their sole carbon sources, making them good candidate species for site remediation.

The last few decades have seen the discovery of a number of bacteria capable of degrading PAHs, particularly low-molecular-weight compounds (e.g., two- and three-ring PAHs such as naphthalene and phenanthrene). Most of these bacteria belong to the genus Agmenellum. Few bacteria are known to degrade higher-molecular-weight PAHs (four- and five-member fused aromatic rings), such as fluoranthene, pyrene, and benzo(a)pyrene; these bacteria include members of the genera Bacillus and Mycobacterium. Degrading high-molecular-weight PAHs is more difficult and generally occurs through co-metabolism during growth on simpler substrates.

A need remains for improving crude oil breakdown by microorganisms and for improving the process of bioremediation through the discovery, enhancement, and application of novel bacterial species that are capable of degrading crude oil components.

### SUMMARY

Certain embodiments are directed to methods of isolating an enhanced crude oil degrading bacterium. In certain aspects, the methods comprise the steps of: (a) selecting a crude oil degrading bacteria and selecting for enhanced crude oil degradation. Selecting an oil degrading bacteria can comprise (i) isolating a crude oil degrading bacteria by culturing a sample containing bacteria with a medium having an initial concentration of crude oil or crude oil derivatives, (ii) introducing the isolated bacteria of (a) to a target environmental condition, and (iii) re-isolating the bacteria isolated in (a) from the target environmental condition. In a further aspect, enhancing the crude oil degrading bacteria can comprise by repeating selection steps (i) to (iii) one or more times using an increased concentration of a target crude oil or crude oil derivative composition during the enhancing process. As additional selections are performed the level of crude oil for selection is increased. The increase need not be incremental or continuous, e.g., the one or more selections can be carried out the same crude oil level with the increase in crude oil level being initiated at some point after 1, 2, 3, 4, or 5 selections.

In certain aspects, the initial concentration of target crude oil is 0.5, 0.75, 1, or 1.5% or more. In certain aspects, the increased concentration or level of crude oil is 1.5, 2, or 3 times greater that the previous crude oil level. The crude oil composition can comprise at least 5, 10, 15, 20, 25, 30, 35, 40, 45, 50, 55, 60, 70, 75, 80, 85, 90, 95, or 100% polyaromatic hydrocarbons (PAH).

In certain embodiments the sample is from a contaminated site. In other embodiments the sample is from a non-contaminated site. In a further aspect, the non-contaminated site is at risk of experiencing a contamination event, such as an oil spill. Such sites include, but are not limited to refineries, beaches, area around pipelines, area around wells and the like.

Certain embodiments are directed to bacterium isolated by the processes described herein. In certain aspects, an isolated crude oil degrading bacterium has a genome comprising a nucleic acid segment that is 90, 92, 94, 96, 98, or 100% identical to one or more sequences selected from SEQ ID NO: 1 to SEQ ID NO:187 (Altogen 322075), or SEQ ID NO:188 to SEQ ID NO:559 (Altogen 635822). In certain aspects the isolated bacterium is Altogen strain 635822 or Altogen strain 322075. In a further aspect, the bacterium is attached to a substrate (e.g., a particle) or comprised in a foam.

Certain embodiments are directed to methods for reducing crude oil contamination comprising contacting a crude oil contaminated site with a bacterium described herein or isolated using a method described herein. In certain aspects, the methods include proactively treating a site at risk of crude oil contamination comprising introducing bacterium described herein or isolated using a method described herein to a non-contaminated site.

Land that is contaminated contains substances in or under the land that are actually or potentially hazardous to health or the environment. Areas with a long history of industrial production will have many sites that may be affected by their former uses such as mining, industry, chemical and oil spills, and waste disposal. These sites are known as Brownfield Land. Contaminated land will have a detectable level of a substance, e.g., crude oil or crude oil derivatives, that is potentially hazardous life and the health of an organism, such as humans or other mammals.

The terms “isolated,” “purified,” or “biologically pure” refer to material that is substantially or essentially free from components that normally accompany it as found in its native state.

The term “providing” is used according to its ordinary meaning “to supply or furnish for use.” In some embodiments, a bacterium is provided directly by contacting a particular site with the bacterium.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. Degradation of Texas crude oil by natural oil-degrading bacteria. (A) Altogen Labs strain 322075 plated on LB agar with 1% tryptone, and (B) control plate with no bacterial culture. Both plates were covered with 2 mm crude oil and incubated at ambient temperature for 15 days.

FIGS. 2A-2B Growth of Altogen strain 635822 on solid agar medium with crude oil. (A) shows the surface of LB agar plate with crude oil following inoculation with and growth of a pure culture of Altogen strain 635822. (B) Surface of LB agar plate with crude oil after incubation with no bacterial inoculation.

FIGS. 3A-3B. Growth of a mixed culture of Altogen strains 322075 and 635822 on solid agar medium with crude oil. (A) shows the surface of LB agar plate with crude oil following inoculation with and growth of a mixed culture prepared from pure cultures of Altogen strains 322075 and 635822. (B) Surface of LB agar plate with crude oil after incubation with no bacterial inoculation.

FIG. 4. Graph showing C18:phytane ratios in crude oil contaminated soil samples, over a period of 25 days following treatment with Altogen strain 322075.

FIG. 5. Graph showing C18:phytane ratios in crude-oil contaminated soil samples, over a period of 25 days following treatment with Altogen strain 635822.

FIG. 6. Graph showing C18:phytane ratios in crude-oil contaminated soil samples, over a period of 25 days following treatment with a mixture of Altogen strains 322075 and 635822.

FIG. 7. Flowchart of method 1 for reactively selecting an enhance bacterium.

FIG. 8. Flowchart of method 2 for proactively selecting an enhance bacterium.

### DESCRIPTION

Bioremediation includes the use of microorganism metabolism to remove pollutants. Technologies can be generally classified as in situ or ex situ. In situ bioremediation involves treating the contaminated material at the site, while ex situ involves the removal of the contaminated material to be treated elsewhere. Microorganisms used to perform the function of bioremediation are known as bioremediators.

The term crude oil as used herein includes crude oil and all liquid, gaseous, and solid (e.g., paraffin) hydrocarbons, or derivative thereof. A “crude oil derivative” refers to a physically or chemically modified crude oil that still retains at least a portion of the crude oil that existed prior to the physical or chemical modification. Crude oil derivatives, therefore, refers to a physically or chemically modified crude oil or crude oil component, including any refined or isolated components or products of crude oil. Such derivatives may have the addition, removal, or substitution of one or more moieties or chemical moieties of the crude oil or a crude oil component. An oil well produces predominantly crude oil, with some natural gas dissolved in it. Because the pressure is lower at the surface than underground, some of the gas will come out of solution and be recovered (or burned) as associated gas or solution gas. A gas well produces predominantly natural gas. However, because the underground temperature and pressure are higher than at the surface, the gas may contain heavier hydrocarbons such as pentane, hexane, and heptane in the gaseous state. At surface conditions these will condense out of the gas to form natural gas condensate, often shortened to condensate. Condensate resembles petrol in appearance and is similar in composition to some volatile light crude oils.

The proportion of light hydrocarbons in the petroleum mixture varies greatly among different oil fields, ranging from as much as 97% by weight in the lighter oils to as little as 50% in the heavier oils and bitumens. The term “light hydrocarbons” refers to C1-C6 hydrocarbons, for example, methanes, ethanes, propanes, butanes, pentane, hexanes, and the like. The hydrocarbons in crude oil are mostly alkanes, cycloalkanes and various aromatic hydrocarbons while the other organic compounds contain nitrogen, oxygen and sulfur, and trace amounts of metals such as iron, nickel, copper and vanadium. The exact molecular composition varies widely from formation to formation but the proportion of chemical elements vary over fairly narrow limits. Thus, the composition of crude oil will vary based on the geographic location from which the crude oil originated.

Described herein are methods of isolating, enhancing, and/or producing bioremediation agents. In certain aspects, the bioremediation agents are selected for a contaminant having a particular chemical profile or classification. The petroleum industry generally classifies crude oil by the geographic location in which it is produced (e.g. West Texas Intermediate, Brent, or Oman), its API gravity (an oil industry measure of density), and its sulfur content. Crude oil may be considered light if it has low density or heavy if it has high density; and it may be referred to as sweet if it contains relatively little sulfur or sour if it contains substantial amounts of sulfur.

The geographic location is important because it affects transportation costs to the refinery. Light crude oil is more desirable than heavy oil since it produces a higher yield of petrol or gasoline, while sweet oil commands a higher price than sour oil because it has fewer environmental problems and requires less refining to meet sulfur standards imposed on fuels in consuming countries. Each crude oil has unique molecular characteristics that are understood by the use of crude oil assay analysis in petroleum laboratories.

### I. ISOLATION OF ENHANCED MICROBIAL STRAINS

In certain embodiments bacteria are selected for degradation of a crude oil or crude oil derivative having a particular composition profile and for environmental robustness. In certain aspects, one or more isolated bacteria are tailored for use in a particular geographic area and/or for a particular contaminated site. A site can be defined by the geographic location and the type and content of material that is contaminated, e.g., a site can be defined as Texas crude oil contaminated soil or beach on the Texas gulf coast. Such a site can be a few square feet to several thousand square feet or more.

The enhancement methods include a positive selection of microbes that degrade crude oil. In certain aspects the crude oil has a composition that is similar to crude oil at a contaminated site. The enhancement process can be carried out prior to a contamination event or during or after such an event. That is the bacteria can be produce as a proactive measure or a reactive measure. Certain embodiments include a first selection at an initial concentration of crude oil. In certain aspects the first selection can be carried out at 0.25, 0.5, 1, 1.5, to 1, 1, 5, 2, 3% crude oil (including all values and ranges there between) or more. In a further aspect, selection can be repeated 1, 2, 3, 4, or more times. The amount of crude oil present in subsequent selection procedures can be increased. The crude oil content in the selection medium can be increased to 2, 2.5, 3, 3.5, 4, to 3, 3.5, 4, 4.5% (including all values and ranges there between) or more.

In another aspect, the bacteria after any of the selection steps can be re-introduced to the site at which the original sample was taken or exposed to an environment that mimics the environment in which the enhanced strains are to be used. This step is to select those bacteria that demonstrate an environmental robustness for the site to be treated. Thus, this step is a negative selection in that those bacteria unable to survive in such an environment are selected against. In certain aspects a molecular marker is used to identify those environmentally robust bacteria that are derived from the bacterial selection process prior to re-introduction or exposure to environmental robustness selection.

Bacteria isolation and selection processes includes “positive selection” where individual bacterial strains with high efficiency oil degradation capabilities are developed, and “negative selection” process when cultures are exposed to environmental stress to enable selection for cells that are efficient oil degraders and thrive in a particular environment. The positive selection process can be repeated with increased percentage of crude oil. In a non-limiting example increased percentages can be 2%, 3%, 4%, 5% or 6% to select for individual strains that provide highest bioremediation capacity. In certain aspects, these selected bacteria will be capable of bioremediation from within a spill and not limited to the periphery of a spill.

Using a reactive positive-negative selection method (i.e., bacteria are selected from a contaminated site) (Method 1) the inventor has successfully isolated two site-specific, oil-degrading bacteria (Altogen Labs strains #322075 (SEQ ID NO:1 to SEQ ID NO:187) and strain #635822 SEQ ID NO:188 to SEQ ID NO:559) from contaminated soil near Galveston, Tex.

The inventor has also developed a proactive positive-negative selection method (Method 2) for generation of highly efficient oil-degrading bacteria for potential oil spills in a particular environment. In certain aspects, the bacteria produced from method 2 can be stock piled in a particular location and dispersed upon a contamination event. In a further aspect, a site that is at risk of contamination can be periodically treated (i.e., a prophylactic treatment) with bacteria produced by method 2 (i.e., a site having little or no significant contamination can exposed to the enhanced bacteria, thus augmenting the natural flora at the contamination site to protect or prepare a site for a potential contamination event).

Method 1—

Contaminated samples are collected from a contaminated site. Samples are collected, for example, into sterile sample bottles and used within about 12, 24, 36, 48 hours or so for microorganism isolation. In certain aspects, samples from contaminated sites may be stored for extended periods until needed. The sample is suspended in 100 ml of distilled water in a sterile glass cylinder, vortexed, and allowed to settle. A portion of the supernatant is inoculated into a growth medium, e.g., LB containing peptone and an initial crude oil content. The flask is incubated at an appropriate temperature (between 30 and 42° C.) for an appropriate amount of time (e.g., 24 to 72 hours) on a rotary shaker at an appropriate speed (e.g., 50 to 200 rpm).

Multiple sub-culturing steps (2, 3, or more) are performed to increase cell number using the same growth medium (e.g., LB containing peptone and initial crude oil content). The growth medium is then centrifuged to collect cell pellets and cell pellets are washed with a sterile solution (e.g., 1×PBS).

Cell pellets are dissolved in mineral salt medium having initial crude oil content. The suspension is then sprayed on an agar plate (Petri dish) having a crude oil film (0.5, 1, 1.5, or 2 mm) absorbed over entire Petri dish surface. Petri dishes are incubated at an appropriate temperature (30 to 42) for an appropriate amount of time to allow colony formation (about 5 to 14 days or more).

Single colonies are picked and inoculated into a growth medium (e.g., LB containing peptone and an initial crude oil content). The flask is incubated at an appropriate temperature (30 to 42 C) for an appropriate time (12 to 72 hours) on a rotary shaker (e.g., at 50 to 200 rpm).

Multiple sub-culturing steps (2, 3, or more) are performed to increase cell number using the same growth medium (e.g., LB containing peptone and an initial crude oil content). The broth is then centrifuged to collect cell pellets and the cell pellets washed with a sterile wash solution.

Cell pellets are dissolved in mineral salt medium having an initial crude oil content. The suspension is used for “negative selection process” by spraying or exposing the suspended cells to target environmental conditions (e.g., oil contaminated soil at the same site where original samples were collected or an artificial environment that mimics a target environmental condition or location).

Sample are collected (e.g., from 7 to 40 days after spraying or exposure) at the environmental site or a mimic thereof and the “positive-negative selection” process repeated with increasing contents of crude oil relative to the initial crude oil content above (e.g., 2, 3, 4, or 5%), followed by the “positive-negative selection” process repeated with the increase in crude oil content. Enhanced bacteria from each selection process are identified by using a molecular marker, e.g., bacteria specific nucleic acid probe, and processed. In certain aspects, a bacterium derived from the original selected bacterium is identified and processed throughout the reminder of the selection process(es).

At the end of two or more selection processes, dry bacterial product is prepared. In certain aspects, the dry bacterial product is prepared at a ratio of 0.0001. 0.001, 0.01, 0.1 to 0.01, 0.05, 0.5. 1 kg of bacteria (including all values and ranges there between) per 100 liters of solution. In one example, the product has 0.2 kg of dry bacteria product suspended in 100 Liters of aqueous solution. This aqueous solution can then be sprayed over contaminated soil (1 gram of dry bacteria product contains approximately 7×10⁸ of bacterial cells). In certain aspects, 100 liters of bacterial product is sprayed per 0.01, 0.1, or 1 acre of contaminated site.

Method 2—

Non-contaminated soil is treated with crude oil (2 mm crude oil film applied) for 2-4 weeks. Non-contaminated soil samples (100 g) are collected from an area at risk for a contamination event (e.g., beaches along known oil shipping routes, refineries, pipelines, well locations and the like). Soil samples are collected into sterile sample bottles and used within about 12, 24, 36, 48 hours or so for microorganism isolation. A sample is suspended in distilled water in sterile glass cylinder, vortexed, and allowed to settle. A portion of the supernatant from upper fraction is inoculated into a growth medium (e.g., LB containing peptone and an initial crude oil content). The growth medium is incubated at an appropriate temperature (30 to 42° C.) for an appropriate time (12-72 hours) on a rotary shaker.

Multiple sub-culturing steps (2, 3, or more) are performed to increase cell number using the same medium (LB containing peptone and an initial crude oil content). The broth is then centrifuged to collect cell pellets and the cell pellets washed with sterile solution (e.g., 1×PBS).

Cell pellets are dissolved in mineral salt medium with an initial crude oil content. The suspension is sprayed on an agar plate (Petri dish) with a crude oil film (0.5, 1, 1.5, or 2 mm) absorbed over entire Petri dish surface. Petri dishes were incubated at an appropriate temperature (30 to 42° C.) for an appropriate amount of time (5 to 14 days) to allow colony formation.

Single colonies are picked up and inoculated into a growth medium (e.g., LB containing peptone and an initial crude oil content). The flask is incubated at an appropriate temperature (30 to 42° C.) for 12 to 72 hours while being aerated (e.g., a rotary shaker at 50 to 200 rpm).

Multiple sub-culturing steps (2, 3, or more) are performed to increase cell number using the same growth medium (e.g., LB containing peptone and an initial crude oil content). The broth is then centrifuged to collect cell pellets and the cell pellets washed with sterile solution (e.g., 1×PBS).

Cell pellets are dissolved in mineral salt medium with an initial crude oil content. The suspension is used for “negative selection process” by spraying or exposing part of the culture to a target environmental condition (e.g., soil at the same site where original samples were collected).

Sample are collected (e.g., from 7 to 40 days after spraying or exposure) at the environmental site or a mimic thereof and the “positive-negative selection” process repeated with increasing contents of crude oil relative to the initial crude oil content above (e.g., 2, 3, 4, or 5%), followed by the “positive-negative selection” process repeated with the increase in crude oil content. Enhanced bacteria from each selection process are identified by using a molecular marker, e.g., bacteria specific nucleic acid probe, and processed. In certain aspects, a bacterium derived from the original selected bacterium is identified and processed throughout the reminder of the selection process(es).

At the end of two or more selection processes, dry bacterial product is prepared. In certain aspects, the dry bacterial product is prepared at a ratio of 0.0001. 0.001, 0.01, 0.1 to 0.01, 0.05, 0.5. 1 kg of bacteria (including all values and ranges there between) per 100 liters of solution. In one example, the product has 0.2 kg of dry bacteria product suspended in 100 Liters of aqueous solution. This aqueous solution can then be sprayed over contaminated soil (1 gram of dry bacteria product contains approximately 7×10⁸ of bacterial cells). In certain aspects, 100 liters of bacterial product is sprayed per 0.01, 0.1, or 1 acre of contaminated site.

### II. BIOREMEDIATION METHODS

Bioremediation is typically defined within the context of biodegradation, a naturally occurring process. Biodegradation is a large component of oil weathering and is a natural process whereby bacteria or other microorganisms alter and break down organic molecules into other substances, eventually producing fatty acids and carbon dioxide. Bioremediation is the acceleration of this process through the addition of exogenous microbial populations, through the stimulation of indigenous populations, or through manipulation of the contaminated media using techniques such as aeration or temperature control (Atlas, Int Biodeterioration & Biodegradation 35(1-13) 317-27, 1995; Hoff, Marine Pollution Bulletin, 26(9):476-81, 1993; Swannell et al., Microbiological Reviews 60(2):342-65, 1996).

Many microorganisms possess the enzymatic capability to degrade petroleum hydrocarbons. Some microorganisms degrade alkanes, others aromatics, and others both paraffinic and aromatic hydrocarbons. Often the normal alkanes in the range C₁₀ to C₂₆ are viewed as the most readily degraded, but low-molecular-weight aromatics, such as benzene, toluene and xylene, which are among the toxic compounds found in crude oil, are also very readily biodegraded by many marine microorganisms.

The biodegradation of crude oil in the marine environment is carried out largely by diverse bacterial populations, including various Pseudomonas species. The hydrocarbon-biodegrading populations are widely distributed in the world's oceans; surveys of marine bacteria indicate that hydrocarbon-degrading microorganisms are ubiquitously distributed in the marine environment.

The initial steps in the biodegradation of hydrocarbons by bacteria and fungi involve the oxidation of the substrate by oxygenases, for which molecular oxygen is required. Alkanes are subsequently converted to carboxylic acids that are further biodegraded via β-oxidation (the central metabolic pathway for the utilization of fatty acids from lipids, which results in formation of acetate which enters the tricarboxylic acid cycle). Aromatic hydrocarbon rings generally are hydroxylated to form diols; the rings are then cleaved with the formation of catechols that are subsequently degraded to intermediates of the tricarboxylic acid cycle.

There are several different bioremediation techniques. The underlying idea is to accelerate the rates of hydrocarbon biodegradation by overcoming the rate-limiting factors. Indigenous populations of microbial bacteria can be stimulated through the addition of nutrients or other materials. Exogenous microbial populations can be introduced in the contaminated environment. The addition of extra bacteria is known as bio-augmentation. In certain aspects, the contaminated media can be manipulated by, for example, aeration or temperature control.

One approach often considered for the bioremediation of crude oil pollutants is the addition of microorganisms (seeding) that are able to degrade hydrocarbons. Most microorganisms considered for seeding are obtained from previously contaminated sites. However, because hydrocarbon-degrading bacteria and fungi are widely distributed in marine, freshwater, and soil habitats, adding seed cultures has proven less promising for treating oil spills than adding fertilizers and ensuring adequate aeration. Most tests have indicated that seed cultures are likely to be of little benefit over the naturally occurring microorganisms at a contaminated site. However, certain aspects described herein are directed to selection methods to increase the likelihood and success of bio-augmentation.

### III. EXAMPLES

The following examples as well as the figures are included to demonstrate preferred embodiments of the invention. It should be appreciated by those of skill in the art that the techniques disclosed in the examples or figures represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods for Isolating Crude Oil-Degrading Microorganisms from Crude Oil Contaminated Sites

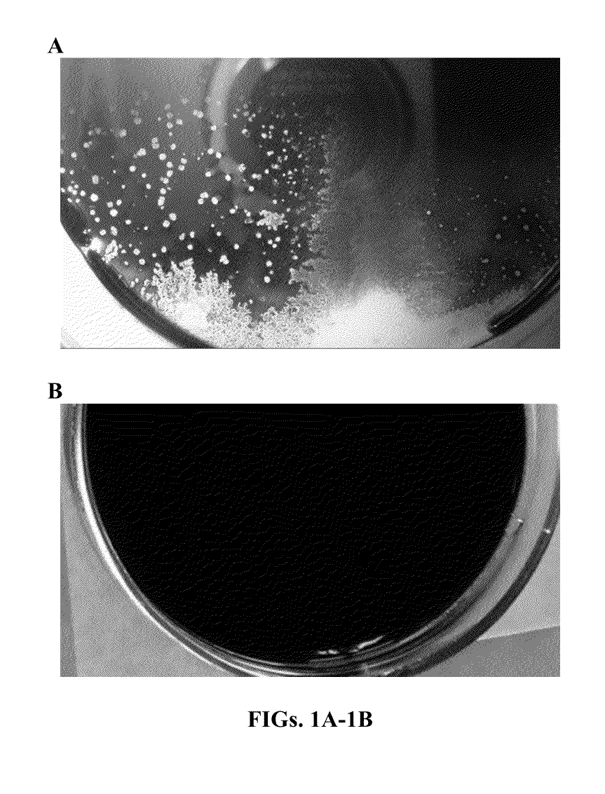

Crude Oil-degrading microorganisms were selected and isolated from a crude oil-contaminated environmental sample. In certain aspects the methods include one or more steps shown in FIG. 1 and described here.

(a) Sample Preparation.

Contaminated soil samples were collected from numerous sites in Galveston, Tex. and Texas City, Tex. Soil samples were collected into sterile sample bottles. The samples were used for microorganism enrichment and isolation within 24 hours. Soil (100 g) was suspended in distilled water (100 ml) in a sterile glass cylinder and vortexed. The suspension was allowed to settle.

(b) Initial Culture in Liquid Growth Medium.

Supernatant (10 ml) from the upper fraction of the settled mixture was removed and used to inoculate sterile LB broth (200 ml) containing peptone and 1% crude oil in a conical flask. The inoculated culture medium was incubated at 32° C. for 48 hrs on a rotary shaker at 100 rpm. Crude oil used in the enrichment steps described here was ‘Texas Crude Oil” obtained from ONTA, Inc. (Toronto, Ontario, CA).

(c) Subcultures in Liquid Growth Medium.

After incubation of the initial culture in liquid growth medium, a portion of the original culture was used to inoculate a second flask having sterile LB broth/peptone/1% crude oil (first subculture), and that flask was incubated as above. A second subculture was prepared with a sample of the first subculture and incubated. Then, a third subculture was prepared with a sample from the second subculture and incubated. All incubations were at 32° C. for 48 hrs on a rotary shaker at 100 rpm. Growth medium in the third subculture was centrifuged at 5,000 rpm for 15 minutes to collect bacteria, and the bacterial cell pellets washed with sterile, 1×PBS solution.

(d) Outgrowth on Solid Growth Medium.

The washed cell pellets from one or more of the subcultures above were resuspended in mineral salts medium (100 ml) (Mittal et al., Ind. J. Exp. Biol., 47:760-765, 2009) having 1% crude oil. The suspension was sprayed onto petri dishes having solid LB medium/agarose (3%) and a 2 mm film of crude oil previously absorbed over their entire surface. Inoculated LB plates were incubated at 32° C. for one week to allow for growth of bacterial colonies.

(e) Outgrowth of Bacterial Colonies in Liquid Growth Medium.

Following colony formation on the solid growth medium, single bacterial colonies were picked and used to inoculate sterile LB broth (200 ml) containing peptone and 1% crude oil in a conical flask. Cultures were incubated as in (b) above.

(f) Second Set of Three Subcultures.

After incubation in Step 5, three additional subcultures were prepared and bacteria were harvested and washed as described above in Step 3.

(g) Reintroduction of Enriched Oil-Degrading Bacteria to Contaminated Site.

Cell pellets from Step 6 were resuspended in 100 ml of mineral salt medium (Mittal et al., 2009) with 1% crude oil. The suspension of bacteria (100 ml) was sprayed onto a region (0.1 acre) (dry bacterial product is prepared using following ratios: 0.2 kg of dry bacteria product suspended in 100 liters of aqueous solution and sprayed over 0.1 acre of contaminated soil (1 gram of dry bacteria product contain 7×10⁸ of bacterial cells) of the contaminated site from which the original soil sample was harvested as described in Step 1 above.

The inventor performed a second round of enrichment for oil-degrading bacteria. Twenty one days following reintroduction of enriched oil-degrading bacteria from the first round of enrichment (Steps 1-6 above), soil samples were again collected from the region of the contaminated site that had been inoculated with bacteria from the first round of enrichment. The soil samples were processed and bacteria were enriched as described in Steps 1-6 above, except that 2% crude oil in the growth media (instead of 1% crude oil) was used throughout the process. Following the second round of enrichment with 2% crude oil, bacteria were prepared as in Step 7, but were suspended in 100 ml of mineral salt medium with 2% crude oil then re-introduced onto the same region of the contaminated site from which the original soil sample was harvested as described in Step 1 above.

The inventor performed a third round of enrichment for oil-degrading bacteria as described for the first and second rounds, except that crude oil was added at 3% to growth media or suspension media as described above.

Following the third round of enrichment at Step 6, the bacteria were harvested, washed with PBS, suspended and plated onto LB agar medium with peptone, previously saturated with crude oil.

Using the methods described in this example, the inventor isolated 32 different bacterial strains from soil samples collected at twelve crude-oil contaminated sites near Galveston, Tex. and Texas City, Tex.

### Example 2

### Degradation of Crude Oil by Bacterial Strains Isolated from an Oil-Contaminated Environmental Site

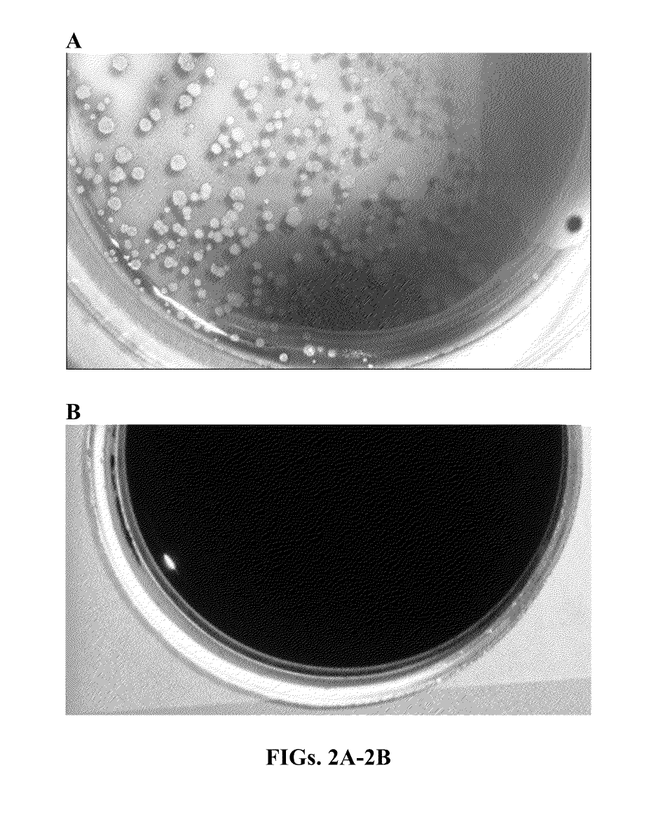

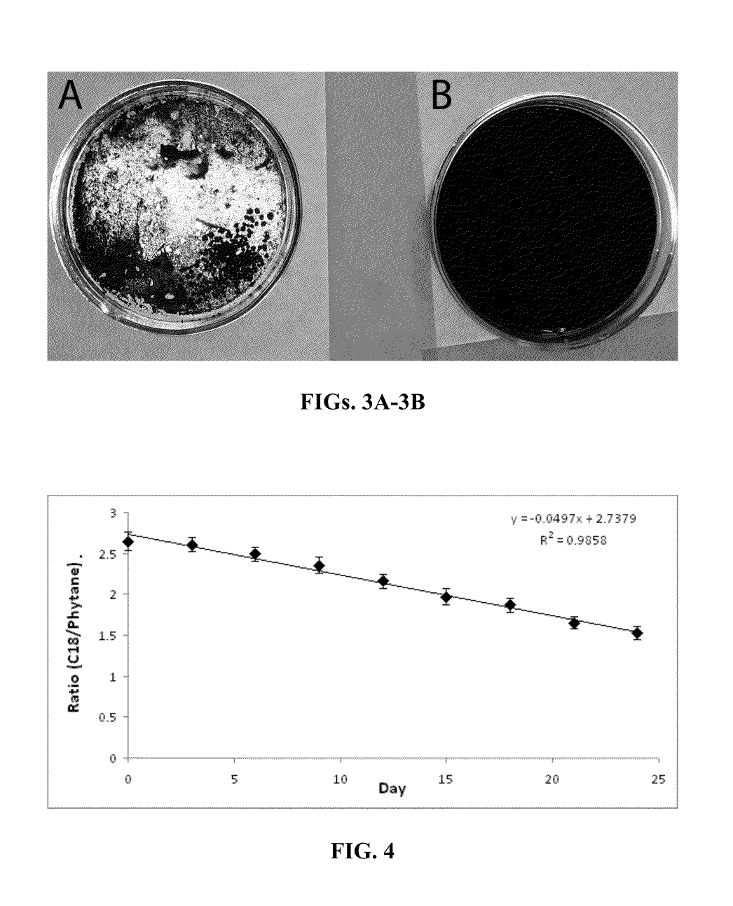

Bacterial strains isolated from oil-contaminated soil were evaluated for their ability to degrade crude oil. Bacterial strains were inoculated separately onto LB agar solid culture medium having 1% tryptone. Each agar plate was covered with a thin (−2 mm) film of “Texas Crude Oil” (ONTA, Inc.; Toronto, Ontario, CA). Agar plates were incubated for 15 days at room temperature and evaluated for oil degradation. Two strains, Altogen Labs 322075 and Altogen Labs 635822, demonstrated the ability to break down crude oil, as indicated by a lightening of the darkly colored crude oil on the surface of the agar medium. FIG. 2 shows LB/tryptone/crude oil agar plates incubated with (FIG. 2A) or without (FIG. 2B) Altogen Labs strain 322075. The agar plate with strain 322075 shows degradation of the crude oil and the control plate with no bacteria exhibited no degradation of the crude oil. FIG. 3 shows LB/tryptone/crude oil agar plates incubated with (FIG. 3A) or without (FIG. 3B) Altogen Labs strain 635822. The agar plate with strain 635822 shows degradation of the crude oil and the control plate with no bacteria exhibited no degradation of the crude oil.

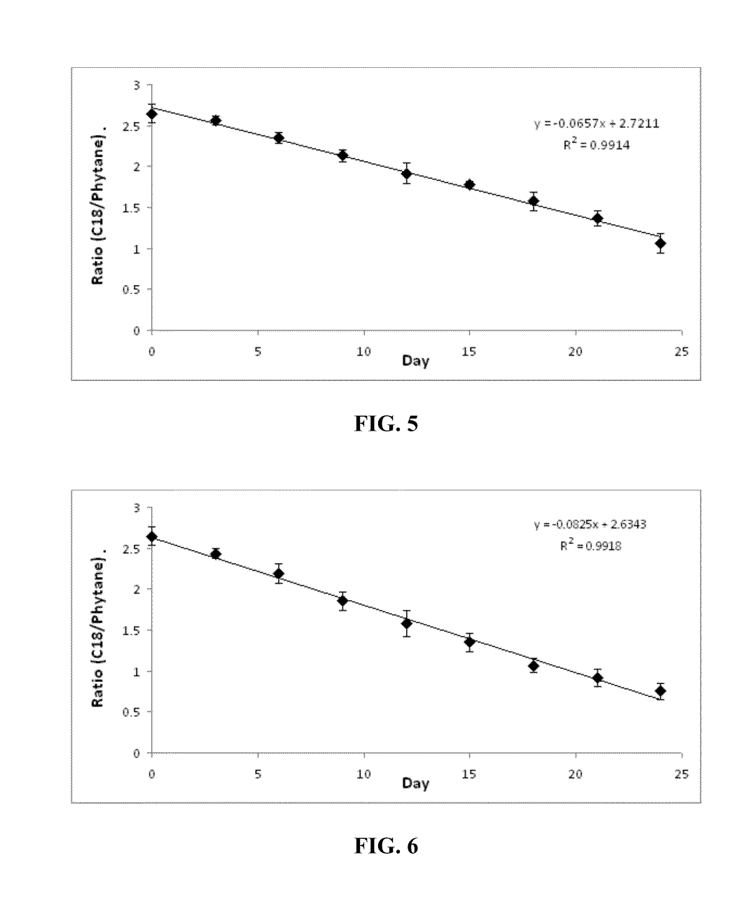

Using the same methods, the two bacterial strains were mixed prior to plating and evaluated for their ability to degrade crude oil as a mixed culture. FIG. 4 shows LB/tryptone/crude oil agar plates incubated with (FIG. 4A) or without (FIG. 4B) a mixture of Altogen Labs strains 322075 and 635822. The culture having the mixture of the two bacterial strains shows extensive degradation of the crude oil and the control plate with no bacteria shows no degradation of the oil.

### Example 3

### Field Test of Crude Oil Degradation by Bacterial Strains of the Invention

Generally, hydrocarbon-degrading microorganisms degrade branched alkanes such as pristane and phytane at much slower rates than their straight chain isomers, C₁₇, heptadecane and C₁₈, octadecane respectively. In contrast, most non-biological processes such as for example physical weathering, volatilization, and leaching do not cause differential losses of normal and branched hydrocarbons that have similar gas chromatographic and chemical behavior. (See Pritchard et al., Biodegradation 3:315-335, 1992)

The inventor used a field test and examined the C₁₈:phytane ratio in treated soil samples following treatment of crude oil-contaminated soil with the bacterial strains individually and with a mixture of the strains. Bacterial cultures were grown in LB broth (200 ml) containing peptone and 3% crude oil in a conical flask. The inoculated culture medium was incubated at 32° C. for 48 hrs on a rotary shaker at 100 rpm. Bacteria were harvested by centrifugation and suspended in an aqueous solution of distilled water having 12% (w/v) sucrose, 0.2% (w/v) agar, and 1.3% (w/v) gelatin, then dried by lyophilization. Dried bacteria (0.2 kg; 1.4×10¹¹ cells) were suspended in 100 L of an aqueous solution. The 100 L of bacterial solution was sprayed over 0.1 acre of crude oil-contaminated soil. The application day was designated Day 0. On days, 3, 6, 9, 12, 15, 18, 21, and 24 post treatment, samples of treated soil were collected and analyzed for C18:phytane ratios in the crude oil by gas chromotography (Mittal et al., 2009).

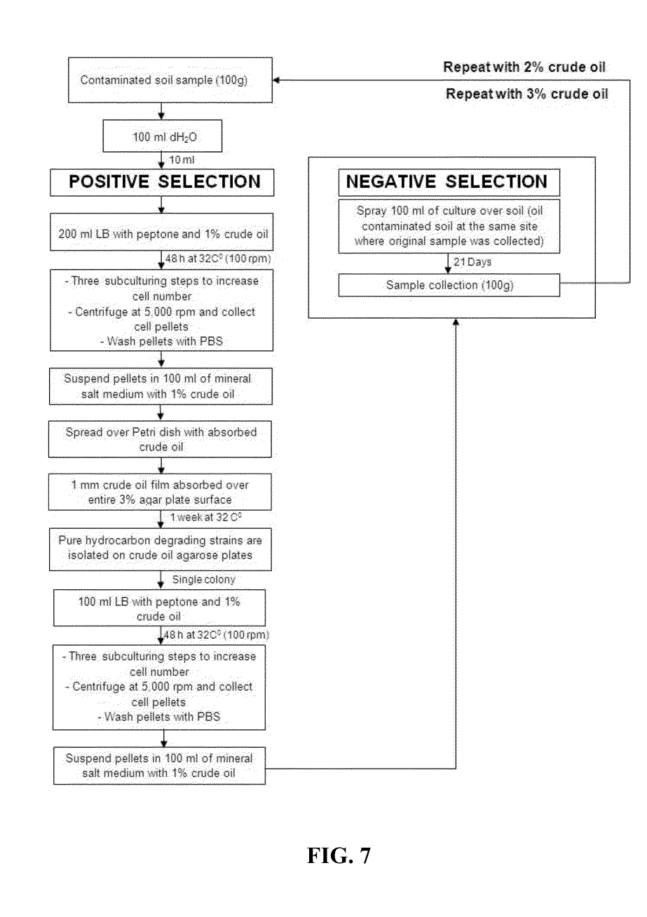

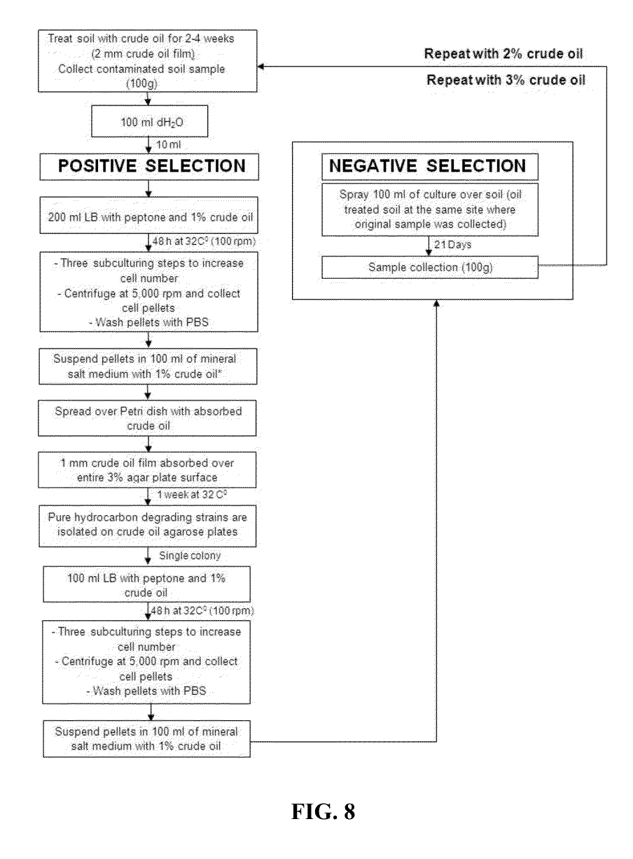

The C18:phytane ratios steadily decreased over the 24 days following treatment with Altogen strain 322075 (FIG. 5), Altogen strain 635822 (FIG. 6), or the mixture of strains 322075 and 635822 (FIG. 7), indicating that the straight chain C₁₈ alkane was degraded at a higher rate than was the branched-chain phytane. The lowes C18:phytane ratio at day 24 was observed in samples that were treated with the mixture of strains.

### Example 4

### 16S rRNA Sequence Analysis of Altogen Strains 322075 and 635822 and Species Identification

The inventor determined the sequences of the 16S rRNAs from Altogen strains 322075 and 635822. The genomic sequences of the strains are represented by the scaffolds that are represented in the sequence listing. A scaffold is a portion of the genome sequence reconstructed from end-sequenced whole-genome shotgun clones. Scaffolds are composed of contigs and gaps. A contig is a contiguous length of genomic sequence in which the order of bases is known to a high confidence level. Gaps occur where reads from the two sequenced ends of at least one fragment overlap with other reads in two different contigs (as long as the arrangement is otherwise consistent with the contigs being adjacent). Since the lengths of the fragments are roughly known, the number of bases between contigs can be estimated. The BLAST algorithm (Altschul et al. 1997) was used to identify closely related bacterial 16S rRNA sequences. Two phylogenetic trees were generated from the sequence relationships between the fifty highest scoring BLAST results. An alignment between these sequences was used to generate a Weighbor-Joining (weighted Neighbor-Joining) phylogenetic tree using the RDP (Ribosomal Database Project) Tree Builder tool at Michigan State University (Cole et al., 2008). All sequences from both trees are part of the Enterobacteriaceae family except for the two species used as outgroups. Both outgroups belong to the Vibrionaceae family, Vibrio mediterranei for the analysis of strain 635822 and Vibrio diazotrophicus for the analysis of strain 322075.

The 16S rRNA sequence determined from Altogen strain 635822 (1,021 nucleotides) was found to be most closely related to that of Serratia fonticola ATCC 29844 (1,490 nucleotides), accession number AJ233429. There was 99.5% sequence similarity and 0.7% ambiguities. The next most closely related species was determined to be S. proteamaculans with a distance score of 1.7%. Based on these data and guidelines, Altogen strain 635822 was determined to be a strain of Serratia fonticola.

The 16S rRNA sequence determined from Altogen strain 322075 (1,010 nucleotides) was found to be most closely related to that of Morganella morganii, ATCC 25830 (1,502 nucleotides), accession number AJ301681. There was 97.8% sequence similarity and 0.9% ambiguity. The next most closely related species was determined to be Providencia rustigianii with a distance score of 3.8%. Based on these data and guidelines, Altogen strain 322075 was identified as a strain of Morganella morganii.

### Example 5

### Genome Sequence Analysis of Serratia Fonticola Strain Altogen 635822 and Morganella MorganII Strain Altogen 322075

The genomes of Altogen strains 635822 and 322075 were determined by a shotgun sequencing method. For each genome, a paired end DNA library was constructed using 5 μg of genomic DNA sheared to an average size of 300 base pairs using a Covaris S2 sonication system (Covaris, Inc., Woburn, Mass., USA). Sheared DNA was treated with a mixture of T4 DNA polymerase, DNA polymerase I, large (Klenow) fragment, and T4 polynucleotide kinase to create blunt-ended DNA. A single adenine base was added to the 3′ end using DNA polymerase I, large (Klenow) fragment (3′ to 5′ exo-) and dATP. A-tailed DNA was ligated with paired end adaptors using T4 DNA ligase. All reagents came from the Paired-End DNA Sample Prep Kit (Illumina, Inc., San Diego, Calif., USA). Size selection of adaptor-ligated DNA was performed by isolation and purification of the target fragment size range (400 bp-450 bp) following agarose gel elctrophoresis of the DNA fragments. The excised fragments were amplified using in-gel PCR with the Phusion® High-Fidelity PCR kit (Illumina, Inc.).

Genome sequencing was performed by Cofactor Genomics (St. Louis, Mo.), USA using the Illumina Genome Analyzer IIx. Cluster generation and sequencing were performed according to the Cluster Station User Guide and Genome Analyzer Operations Guide provided by Illumina. Primary data (sequencing reads) were generated using the Illumina Pipeline version SCS 2.8.0 paired with OLB 1.8.0. The configuration file used as input to Illumina's GERALD software in the GAPipeline 1.5.1 for fragment runs was “USE_BASES Y*, SEQUENCE_FORMAT, —fastq, ANALYSIS sequence”. The configuration used for paired end runs was “USE_BASES Y*,Y*, SEQUENCE_FORMAT, —fastq, ANALYSIS sequence_pair”. Novoalign version 2.07.05 (Novocraft Technologies; Selangor, Malaysia) was used for all sequence alignments. Alignment parameters were: “-o SAM -F ILMFQ -r all-130-t 140-e 10”. The Cofactor Genomics (St. Louis, Mo., USA) assembly software pipeline is optimized to obtain the most contiguous assembly by filtering low occurrence observations. The software uses SOAPdenovo 1.05 for assembling the ILMN reads and determines the optimal assembly for each respective dataset.

The genome sequence determined for Serratia fonticola strain Altogen 635822 had a total assembly length of 5,681,221 bp in 185 scaffolds. For comparison, the genome sizes of four closely related organisms are (1) Serratia sp. AS13; 5,442,549 bp, (2) Serratia sp. AS12; 5,443,009 bp, (3) Serratia sp. AS9; 5,442,880 bp, and (4) Serratia proteamaculans 568; 5,448,853 bp.

The genome sequence determined for Morganella morganii strain Altogen 322075 had a total assembly length of 4,098,746 bp in 152 scaffolds. No complete genome sequences from closely related species were found to be publicly available for comparison.

For annotation of sequences, genome sequencing assemblies were uploaded to the automated annotation platform Rapid Annotation using Subsystems Technology (RAST) server maintained by the National Microbial Pathogen Data Resource (Aziz et al., BMC Genomics 9:75, 2008). For Altogen strain 635822, the genome sequence was found to have 5,078 protein encoding genes and 49 RNA encoding sequences. For Altogen strain 322075, the genome sequence was found to have 3,915 protein encoding genes 48 RNA encoding sequences. A single rRNA-encoding sequence was found for each organism.

### Example 6

### Genomic and Proteomic Analysis of Serratia Fonticola Strain Altogen 63582′2 and Morganella MorganII Strain Altogen 322075

Analyses of the genome sequences of Altogen strains 2635822 and 322075 were performed to identify potential genes and proteins that have significant sequence identity or similarity to genes and proteins that are known to be involved in petroleum oil degradation pathways or biosurfactant production pathways in other bacteria. Gene sequence data from related species were gathered from the literature, SEED Viewer (Overbeek et al., Nucleic Acids Res. 33:17, 2005) and the NCBI Biosystems database (Geer et al., Nucleic Acids Res. 38 (database issue):D492-D496, Epub 2009 Oct. 23, 2010). Subsequently, these sequences were used in a BLAST+ (blastp) query against a local database of the genome sequences from Altogen strains 635822 and 322075.

Biosurfactant associated proteins predicted to be encoded by genes from Altogen strain 635822 or strain 322075 and having significant sequence identity with similar proteins known to be produced by different bacterial species are shown in Table 1.

The biosurfactant, serrawettin W1, is produced by Serratia marcescens and its synthesis involves genes including pswP (phosphopantetheinyl transferase), swrW (unimodular synthetase, a nonribosomal peptide synthetase (NRPS)), and hexS (LysR-type transcriptional regulator). The proteins encoded by these genes were found to have significant sequence identity with predicted proteins encoded by similar genes in Altogen strain 635822 (Table 1). Altogen strain 322075 possesses a gene encoding a protein with significant sequence identity to HexS. HexS has been shown to simultaneously down-regulate swrW and prodigiosin synthesis while leaving pswP unaffected (Tanikawa et al., Microbiology and Immunolgy, 50:587-596, 2006). Genes encoding the prodigiosin synthesis pathway, starting with the pigA gene, are present in both Altogen strains (data not shown).

Quorum sensing systems are also known to regulate biosurfactant production. In Serratia liquefaciens, the quorum sensing genes swrI and swrR and N-acylhomoserine lactones (AHLs) have been implicated in the regulation of biosurfactants. Proteins encoded by swrI, swrR and AHL synthesis genes were found to have significant sequence identity with predicted proteins encoded by similar genes in Altogen strain 635822 (Table 1).

Serrawettin activity is generally temperature dependent in species of Serratia. Temperature dependent proteins derived from the genes tdrA were found to have significant sequence identity with predicted proteins encoded by similar genes in Altogen strains 635822 and 322075 (Table 1). Another temperature dependent protein, encoded by yhcR, has significant identity with a predicted protein encoded by a gene in Altogen strain 635822.

The bioemulsifier alasan found in species of Acinetobacter is a complex of a polysaccharide and the proteins AlnA, AlnB and AlnC. Both Altogen strains were found to have genes encoding predicted proteins similar to OmpA-like protein precursors involved in alasan biosynthesis. Furthermore, both strains also had genes encoding predicted proteins similar to AlnB Although AlnB by itself is not known to have emulsifying activity, it is known to stabilize oil-in-water emulsions generated by the OmpA-like protein AlnA

Rhamnolipids are well characterized bacterial surfactants. Both Altogen strains have genes encoding predicted proteins with significant sequence identity to known proteins involved in the rhamnolipid synthesis pathway (Table 1).

TABLE 1
Predicted biosurfactant-related proteins encoded by genes present in Altogen strain
635822 and Altogen strain 322075 are shown. Acc. No., accession number.

| | | | | | |
|---|---|---|---|---|---|
| RAST Protein | | | | | |
| Prediction for | | Bacterial | BLAST | % | |
| Altogen Strain | Protein | Species | E-Value | Identity | Acc. No. |

Biosurfactant Regulators - Strain 635822

| | | | | | |
|---|---|---|---|---|---|
| transcriptional | transcriptional | Serratia | 7e−160 | 85 | BAD11811.1 |
| regulator IrhA | regulator HexS | marcescens | | | |
| | (hexS) | | | | |
| probable | hypothetical | Serratia | 9e−31 | 94 | BAB85653.1 |
| membrane | protein for | marcescens | | | |
| protein YPO3684 | temperature | | | | |
| | dependent | | | | |
| | production (yhcR) | | | | |
| LysR family | putative | Serratia | 1e−163 | 91 | BAB84544.1 |
| transcriptional | temperature- | marcescens | | | |
| regulator QseA | dependent | | | | |
| | regulator A (tdrA) | | | | |

Biosurfactant Regulators - Strain 322075

| | | | | | |
|---|---|---|---|---|---|
| LysR family | LysR-family | Serratia | 7e−106 | 63 | BAD11811.1 |
| transcriptional | transcriptional | marcescens | | | |
| regulator IrhA | regulator HexS | | | | |
| | (hexS) | | | | |
| transcriptional | putative | Serratia | 2e−40 | 33 | BAB84544.1 |
| regulator, LysR | temperature- | marcescens | | | |
| family | dependent | | | | |
| | regulator A (tdrA) | | | | |

Biosurfactants - Strain 635822

| | | | | | |
|---|---|---|---|---|---|
| 4′-phosphopant- | putative 4′- | Serratia | 3e−78 | 62 | BAD11765.1 |
| etheinyl | phosphopant- | marcescens | | | |
| transferase (EC | etheinyl | | | | |
| 2.7.8.—) | transferase | | | | |
| [enterobactin] | (pswP) | | | | |
| siderophore | | | | | |
| Enterobactin | putative | Serratia | 6e−155 | 33 | BAD60917.1 |
| synthetase | serrawettin W1 | marcescens | | | |
| component F, | synthetase (swrW) | | | | |
| serine activating | | | | | |
| enzyme (EC | | | | | |
| 2.7.7.—) | | | | | |
| Alkyl | Peroxiredoxin, | Acinetobacter | 3e−29 | 38 | AAS77881.1 |
| hydroperoxide | alasan | radioresistens | | | |
| reductase subunit | biosynthesis | | | | |
| C-like protein | (alnB) | | | | |
| putative | OmpA-like protein | Acinetobacter | 5e−14 | 41 | AAK57731.1 |
| lipoprotein | precursor, alasan | radioresistens | | | |
| | biosynthesis | | | | |

Blosurfactants - Strain 32205

| | | | | | |
|---|---|---|---|---|---|
| Putative outer | OmpA-like protein | Acinetobacter | 7e−11 | 34 | AAK57731.1 |
| membrane | precursor, alasan | radioresistens | | | |
| lipoprotein | biosynthesis | | | | |
| Alkyl | Peroxiredoxin, | Acinetobacter | 2e−82 | 75 | AAS77881.1 |
| hydroperoxide | alasan | radioresistens | | | |
| reductase protein | biosynthesis | | | | |
| C (EC 1.6.4.—) | (alnB) | | | | |

Quorum Sensing - Strain 635822

| | | | | | |
|---|---|---|---|---|---|
| Quorum-sensing | SwrR (swrR) | Serratia | 6e−62 | 46 | AAO38761.1 |
| transcriptional | | liquefaciens | | | |
| activator YspR | | | | | |
| N-3-oxooctanoyl- | SwrI (swrI) | Serratia | 8e−62 | 69 | AAB18141.1 |
| L-homoserine | | liquefaciens | | | |
| lactone synthase; | | | | | |
| N-3-oxohexanoyl- | | | | | |
| L-homoserine | | | | | |
| lactone synthase | | | | | |
| Regulatory | putative quorum- | Serratia | 5e−15 | 32 | ZP_08039640.1 |
| protein RecX | sensing regulatory | symbiotica str. | | | |
| | protein (spnT) | Tucson | | | |
| Homoserine | SplI (splI) | Serratia | 4e−44 | 40 | AAR32908.1 |
| lactone synthase | | plymuthica RVH1 | | | |
| YpeI | | | | | |

Quorum Sensing - Strain 322075

| | | | | | |
|---|---|---|---|---|---|
| Regulatory | putative quorum- | Serratia | 2e−29 | 42 | ZP_08039640.1 |
| protein recX | sensing regulatory | symbiotica str. | | | |
| (oraA protein) | protein (spnT) | Tucson | | | |

Rhamnolipid Synthesis - Strain 635822

| | | | | | |
|---|---|---|---|---|---|
| Phosphomanno- | Phosphomanno- | Pseudomonas | 2e−52 | 33 | P26276.4 |
| mutase (EC | mutase/ | aeruginosa | | | |
| 5.4.2.8) | phosphogluco- | PAO1 | | | |
| | mutase, PMM/ | | | | |
| | PGM (algC) | | | | |
| 3-oxoacyl-[acyl- | rhamnolipid | Mycobacterium | 1e−30 | 38 | YP_884537.1 |
| carrier protein] | biosynthesis 3- | smegmatis str. | | | |
| reductase (EC | oxoacyl-ACP | MC2 155 | | | |
| 1.1.1.100) | reductase | | | | |
| 2-deoxy-D- | NADPH- | Pseudomonas | 7e−29 | 34 | AAD53514.1 |
| gluconate 3- | dependent beta- | aeruginosa | | | |
| dehydrogenase | ketoacyl reductase | PAO1 | | | |
| (EC 1.1.1.125) | (rhlG) | | | | |
| Putative lipase | Esterase EstA, | Pseudomonas | 2e−27 | 27 | O33407.1 |
| | Autotransporter | aeruginosa | | | |
| | esterase EstA | PAO1 | | | |
| | (estA or papA) | | | | |
| Septum site- | MotR | Pseudomonas | 3e−12 | 27 | AAC62540.2 |
| determining | | aeruginosa | | | |
| protein MinD | | | | | |
| COG1720: | RcsF (rcsF) | Pseudomonas | 5e−51 | 51 | AAD53515.1 |
| Uncharacterized | | aeruginosa | | | |
| conserved protein | | PAO1 | | | |
| dTDP-rhamnosyl | rhamnosyltransferase- | Pseudomonas | 6e−23 | 29 | CBI71058.1 |
| transferase RfbF | 2 (rhlC) | aeruginosa | | | |
| (EC 2.—.—.—) | | | | | |
| Cyclohexadienyl | Cyclohexadienyl | Pseudomonas | 4e−68 | 47 | Q01269.2 |
| dehydratase (EC | dehydratase, | aeruginosa | | | |
| 4.2.1.51)(EC | Prephenate | PAO1 | | | |
| 4.2.1.91) | dehydratase, | | | | |
| | Arogenate | | | | |
| | dehydratase | | | | |
| | (pheC) | | | | |

Rhamnolipid Synthesis - Strain 322075

| | | | | | |
|---|---|---|---|---|---|
| Phosphoglucos- | Phosphomanno- | Pseudomonas | 2e−25 | 27 | P26276.4 |
| amine mutase | mutase/ | aeruginosa | | | |
| (EC 5.4.2.10) | phosphogluco- | PAO1 | | | |
| | mutase, PMM/ | | | | |
| | PGM (algC) | | | | |
| 3-oxoacyl-[acyl- | rhamnolipid | Mycobacterium | 8e−24 | 33 | YP_884537.1 |
| carrier protein] | biosynthesis 3- | smegmatis str. | | | |
| reductase (EC | oxoacyl-ACP | MC2 155 | | | |
| 1.1.1.100) | reductase | | | | |
| 3-oxoacyl-[acyl- | NADPH- | Pseudomonas | 6e−29 | 33 | AAD53514.1 |
| carrier protein] | dependent beta- | aeruginosa | | | |
| reductase (EC | ketoacyl reductase | PAO1 | | | |
| 1.1.1.100) | (rhlG) | | | | |
| Septum site- | MotR | Pseudomonas | 3e−12 | 25 | AAC62540.2 |
| determining | | aeruginosa | | | |
| protein MinD | | | | | |
| COG1720: | RcsF (rcsF) | Pseudomonas | 3e−49 | 49 | AAD53515.1 |
| Uncharacterized | | aeruginosa | | | |
| conserved protein | | PAO1 | | | |
| Cystine ABC | Cyclohexadienyl | Pseudomonas | 8e−15 | 27 | Q01269.2 |
| transporter, | dehydratase, | aeruginosa | | | |
| periplasmic | Prephenate | PAO1 | | | |
| cystine-binding | dehydratase, | | | | |
| protein FliY | Arogenate | | | | |
| | dehydratase | | | | |
| | (pheC) | | | | |

Both Altogen strains were analyzed for gene sequences and predicted protein sequences that may be involved in crude oil degradation pathways. Proteins involved in hydrocarbon metabolism that are predicted to be encoded from genes present in Altogen strain 635822 or strain 322075 and that have significant sequence identity with similar proteins known to be produced by different bacterial species are shown in Table 2.

Protocatechuate dioxygenases are known to cleave aromatic rings such as those present in PAHs. Benzene, toluene, xylenes, phenol, naphthalene, and biphenyl are among a group of compounds that have at least one reported pathway for biodegradation involving catechol dioxygenase enzymes. Thus, detection of the corresponding catechol dioxygenase genes can serve as a basis for identifying bacteria that have these catabolic abilities. Sequence analyses revealed that the protocatechuate 4,5-dioxygenase protein (extradiol catechol dioxygenase that catalyzes the oxidative cleavage of substituted catechols; part of the bacterial aromatic compound degradation pathway) produced by Serratia proteamaculans has significant sequence identity with a predicted protein encoded by a gene in Altogen strain 635822 Table 2.

Serratia proteamaculans 568 produces three proteins used in the degradation of aromatic compounds. The enzyme 4-hydroxy-2-ketovalerate aldolase catalyzes the formation of pyruvate and acetaldehyde from 4-hydroxy-2-ketovaleric acid and is involved in the degradation of phenylpropionate. The enzyme 4-hydroxy-2-oxovalerate aldolase, encoded by the gene mhpE, is also involved in the catabolism of phenylpropionate. Beta alanine pyruvate transaminase catalyzes the formation of pyruvate and beta-alanine from L-alanine and 3-oxopropanoate. Both Altogen strains were found to have genes encoding predicted proteins with sequence identity to each of these three proteins (Table 2).

Rubredoxin and rubredoxin reductase proteins are known to have roles in alkane metabolism. A rubredoxin reductase from Acinetobacter has sequence identity with a predicted protein encoded by a gene from Altogen strain 635822 (data not shown). Two predicted proteins, rubredoxin and rubredoxin reductase, encoded by genes in Altogen strain 322075, were found to have significant sequence identity with proteins produced by Acinetobacter sp. ADP1 (Table 2).

TABLE 2
Predicted hydrocarbon degradation pathway proteins encoded by genes present in
Altogen strain 635822 and Altogen strain 322075 are shown. Acc. No., accession number.

| | | | | | |
|---|---|---|---|---|---|
| RAST Protein | | | | | |
| Prediction for | | Bacterial | BLAST | % | |
| Altogen Strain | Protein | Species | E-Value | Identity | Acc. No. |

Aromatic Hydrocarbon Degradation - Strain 635822

| | | | | | |
|---|---|---|---|---|---|
| 2,3-dihydroxy- | protocatechuate | Serratia | 7e−11 | 27 | YP_001478327.1 |
| phenylpropionate | 4,5-dioxygenase | proteamaculans | | | |
| 1,2-dioxygenase | | | | | |
| (EC 1.13.11.—) | | | | | |
| Putative O- | putative O- | Yersinia | 4e−140 | 65 | YP_001005235.1 |
| methyltransferase | methyltransferase | enterocolitica | | | |
| | | subsp. | | | |
| | | enterocolitica | | | |
| | | 8081 | | | |
| 2,3-dihydroxy- | 2,3-dihydroxy-2,3- | Pseudomonas | 5e−47 | 42 | YP_534824.1 |
| 2,3-dihydro- | dihydro- | putida | | | |
| phenylpropionate | phenylpropionate | | | | |
| dehydrogenase | dehydrogenase | | | | |
| (EC 1.3.1.—) | (nahB) | | | | |
| Dihydrodipico- | Trans-O- | Pseudomonas | 4e−12 | 25 | P0A144.1 |
| linate synthase | hydroxybenzyl- | putida | | | |
| (EC 4.2.1.52) | idenepyruvate | | | | |
| | hydratase- | | | | |
| | aldolase (nahE) | | | | |
| Long-chain fatty | aromatic | Serratia sp. AS12 | 0 | 80 | YP_004501990.1 |
| acid transport | hydrocarbon | | | | |
| protein | degradation | | | | |
| | membrane protein | | | | |
| 2,4- | 2,4-dihydroxyhept- | Serratia sp. AS13 | 1e−137 | 89 | AEG26382.1 |
| dihydroxyhept-2- | 2-ene-1,7-dioic | | | | |
| ene-1,7-dioic acid | acid aldolase | | | | |
| aldolase (EC | | | | | |
| 4.1.2.—) | | | | | |
| 5-carboxymethyl- | 4-hydroxyphenyl- | Serratia sp. AS13 | 4e−139 | 92 | AEG26377.1 |
| 2-oxo-hex-3-ene- | acetate degradation | | | | |
| 1,7-dioate | bifunctional | | | | |
| decarboxylase | isomerase/de- | | | | |
| (EC 4.1.1.68)/2- | carboxylase, HpaG | | | | |
| hydroxyhepta- | 2 subunit | | | | |
| 2,4-diene-1,7- | | | | | |
| dioate isomerase | | | | | |
| (EC 5.3.3.—) | | | | | |
| 2-oxo-hepta-3- | 2-oxo-hepta-3- | Serratia sp. AS13 | 9e−153 | 96 | AEG26381.1 |
| ene-1,7-dioic acid | ene-1,7-dioic acid | | | | |
| hydratase (EC | hydratase | | | | |
| 4.2.—.—) | | | | | |
| 5-carboxymethyl- | 4-hydroxyphenyl- | Serratia sp. AS13 | 4e−139 | 92 | AEG26376.1 |
| 2-oxo-hex-3-ene- | acetate | | | | |
| 1,7-dioate | degradation | | | | |
| decarboxylase | bifunctional | | | | |
| (EC 4.1.1.68)/2- | isomerase/de- | | | | |
| hydroxyhepta- | carboxylase, | | | | |
| 2,4-diene-1,7- | HpaG1 subunit | | | | |
| dioate isomerase | | | | | |
| (EC 5.3.3.—) | | | | | |
| Transcriptional | transcriptional | Serratia sp. AS13 | 3e−160 | 89 | AEG26384.1 |
| activator of 4- | regulator, AraC | | | | |
| hydroxyphenyl- | family | | | | |
| acetate 3- | | | | | |
| monooxygenase | | | | | |
| operon, | | | | | |
| XylS/AraC family | | | | | |
| 3-dehydroquinate | 3-dehydroquinate | Serratia sp. AS13 | 2e−69 | 93 | AEG30273.1 |
| dehydratase II | dehydratase | | | | |
| (EC 4.2.1.10) | | | | | |
| Phenylacetaldehyde | Phenylacetaldehyde | Serratia sp. AS13 | 0 | 90 | AEG26721.1 |
| dehydrogenase | dehydrogenase | | | | |
| (EC 1.2.1.39) | | | | | |
| 3,4- | 3,4- | Serratia | 6e−164 | 94 | ABV39734.1 |
| dihydroxyphenylacetate | dihydroxyphenylacetate | proteamaculans | | | |
| 2,3- | 2,3- | 568 | | | |
| dioxygenase (EC | dioxygenase | | | | |
| 1.13.11.15) | | | | | |
| 4-hydroxyphenyl- | 4-hydroxyphenyl- | Serratia | 0 | 92 | ABV39738.1 |
| acetate | acetate | proteamaculans | | | |
| symporter, major | transporter | 568 | | | |
| facilitator | | | | | |
| superfamily | | | | | |
| (MFS) | | | | | |
| 5-carboxymethyl- | 5-carboxymethyl- | Serratia | 4e−65 | 87 | ABV39735.1 |
| 2-hydroxymuconate | 2-hydroxymuconate | proteamaculans | | | |
| delta- | isomerase | 568 | | | |
| isomerase (EC | | | | | |
| 5.3.3.10) | | | | | |
| 5-carboxymethyl- | 5-carboxymethyl-2 | Serratia | 0 | 98 | ABV39733.1 |
| 2 hydroxy-muconate | hydroxy-muconate | proteamaculans | | | |
| semialdehyde | semialdehyde | 568 | | | |
| dehydrogenase | dehydrogenase | | | | |
| (EC 1.2.1.60) | | | | | |
| Benzoate | benzoate | Serratia | 0 | 86 | ABV41331.1 |
| transport protein | transporter | proteamaculans | | | |
| | | 568 | | | |
| Aldehyde | phenylacetic acid | Serratia | 0 | 90 | ABV42172.1 |
| dehydrogenase | degradation | proteamaculans | | | |
| (EC 1.2.1.3), | protein paaN | 568 | | | |
| PaaZ | | | | | |
| Esterase ybfF | alpha/beta | Pectobacterium | 8e−102 | 69 | YP_003016794.1 |
| (EC 3.1.—.—) | hydrolase fold | carotovorum | | | |
| | protein | subsp. | | | |
| | | cartovorum PC1 | | | |
| Acetaldehyde | acetaldehyde | Klebsiella | 2e−147 | 84 | ACI11884.1 |
| dehydrogenase, | dehydrogenase | pneumoniae 342 | | | |
| acetylating, (EC | (mhpF) | | | | |
| 1.2.1.10) in gene | | | | | |
| cluster for | | | | | |
| degradation of | | | | | |
| phenols, cresols, | | | | | |
| catechol | | | | | |
| Monoamine | Primary-amine | Enterobacter | 0 | 79 | ADO48675.1 |
| oxidase (1.4.3.4) | oxidase | cloacae SCF1 | | | |
| 3-phenylpropionate | 3-phenylpropionate | Escherichia coli | 1e−20 | 76 | CBG35577.1 |
| dioxygenase | dioxygenase | 042 | | | |
| ferredoxin subunit | ferredoxin subunit | | | | |
| | (hcaC) | | | | |
| 3-phenylpropionate | 3-phenylpropionate | Escherichia coli | 3e−110 | 55 | CBG35579.1 |
| dioxygenase | dioxygenase | 042 | | | |
| ferredoxin-- | ferredoxin-- | | | | |
| NAD(+) | NAD(+) reductase | | | | |
| reductase | component (hcaD) | | | | |
| component (EC | | | | | |
| 1.18.1.3) | | | | | |
| Transcriptional | Transcriptional | Salmonella | 1e−61 | 80 | P40676.3 |
| regulator SlyA | regulator slyA, | enterica subsp. | | | |
| | Cytolysin slyA, | enterica serovar | | | |
| | Salmolysin (slyA) | Typhimurium | | | |
| Oxidoreductase | 3-hydroxyisobutyrate | Pseudomonas | 6e−43 | 40 | P28811.1 |
| YihU | dehydrogenase | aeruginosa PAO1 | | | |
| | (mmsB) | | | | |
| 2-octaprenyl-3- | 3-(3-hydroxy- | Escherichia coli | 3e−13 | 27 | P77397.1 |
| methyl-6- | phenyl)propionate/ | K-12 | | | |
| methoxy-1,4- | 3- | | | | |
| benzoquinol | hydroxycinnamic | | | | |
| hydroxylase (EC | acid hydroxylase | | | | |
| 1.14.13.—) | (mhpA) | | | | |
| Phenylacetate- | oxygenase | Rhodococcus | 2e−47 | 33 | AAL96830.1 |
| CoA | reductase KshB | erythropolis | | | |
| oxygenase/reductase, | (kshB) | | | | |
| PaaK | | | | | |
| subunit | | | | | |
| 4-hydroxy-2- | 4-hydroxy-2- | Serratia | 6e−65 | 42 | YP_001479254.1 |
| oxovalerate | ketovalerate | proteamaculans | | | |
| aldolase (EC | aldolase | 568 | | | |
| 4.1.3.—) | | | | | |
| Adenosylmethionine- | beta alanine-- | Serratia | 5e−46 | 31 | YP_001476841.1 |
| 8-amino-7- | pyruvate | proteamaculans | | | |
| oxononanoate | transaminase | 568 | | | |
| aminotransferase | | | | | |
| (EC 2.6.1.62) | | | | | |

Aromatic Hydrocarbon Degradation - Strain 322075

| | | | | | |
|---|---|---|---|---|---|
| Lysine-N- | flagellin lysine-N- | Yersinia | 4e−50 | 29 | YP_001006727.1 |
| methylase (EC | methylase (fliB) | enterocolitica | | | |
| 2.1.1.—) | | subsp | | | |
| | | enterocolitica | | | |
| | | 8081 | | | |
| O- | hydroxyneurosporene- | Enterobacter sp. | 2e−71 | 39 | YP_001176654.1 |
| demethylpuromycin- | O- | 638 | | | |
| O- | methyltransferase | | | | |
| methyltransferase | | | | | |
| (EC 2.1.1.38) | | | | | |
| Short-chain | 2,3-dihydroxy-2,3- | Pseudomonas | 1e−19 | 30 | YP_534824.1 |
| dehydrogenase/reductase | dihydrophenylpropionate | putida | | | |
| SDR | dehydrogenase | | | | |
| | (nahB) | | | | |
| Long-chain fatty | aromatic | Serratia sp. AS12 | 8e−155 | 60 | YP_004501990.1 |
| acid transport | hydrocarbon | | | | |
| protein | degradation | | | | |
| | membrane protein | | | | |
| FIG094199: | 4- | Serratia sp. AS13 | 2e−29 | 34 | AEG26377.1 |
| Fumarylacetoacetate | hydroxyphenylacetate | | | | |
| hydrolase | degradation | | | | |
| | bifunctional | | | | |
| | isomerase/decarboxylase, | | | | |
| | HpaG2 | | | | |
| | subunit | | | | |
| transcriptional | transcriptional | Serratia sp. AS13 | 2e−12 | 37 | AEG26384.1 |
| regulator, AraC | regulator, AraC | | | | |
| family | family | | | | |
| 3-dehydroquinate | 3-dehydroquinate | Serratia sp. AS13 | 7e−58 | 80 | AEG30273.1 |
| dehydratase II | dehydratase | | | | |
| (EC 4.2.1.10) | | | | | |
| Nitrate/nitrite | 4- | Serratia | 5e−62 | 31 | ABV39738.1 |
| transporter | hydroxyphenylacetate | proteamaculans | | | |
| | transporter | 568 | | | |
| Aldehyde | 5-carboxymethyl- | Serratia | 1e−98 | 38 | ABV39733.1 |
| dehydrogenase B | 2- | proteamaculans | | | |
| (EC 1.2.1.22) | hydroxymuconate | 568 | | | |
| | semialdehyde | | | | |
| | dehydrogenase | | | | |
| Benzoate | benzoate | Serratia | 2e−117 | 61 | ABV41331.1 |
| transport protein | transporter | proteamaculans | | | |
| | | 568 | | | |
| Esterase ybfF | alpha/beta | Pectobacterium | 3e−88 | 63 | YP_003016794.1 |
| (EC 3.1.—.—) | hydrolase fold | carotovorum | | | |
| | protein | subsp. | | | |
| | | carotovorum PC1 | | | |
| Transcriptional | Transcriptional | Escherichia coli | 2e−48 | 67 | P40676.3 |
| regulator SlyA | regulator slyA, | 042 | | | |
| | Cytolysin slyA | | | | |
| | (slyA) | | | | |
| NADH | oxygenase | Rhodococcus | 9e−27 | 28 | AAL96830.1 |
| oxidoreductase | reductase KshB | erythropolis | | | |
| hcr (EC 1.—.—.—) | (kshB) | | | | |
| 2-isopropylmalate | 4-hydroxy-2- | Serratia | 2e−14 | 26 | YP_001479254.1 |
| synthase (EC | ketovalerate | proteamaculans | | | |
| 2.3.3.13) | aldolase | 568 | | | |
| Adenosylmethionine- | beta alanine-- | Serratia | 5e−43 | 29 | YP_001476841.1 |
| 8-amino-7- | pyruvate | proteamaculans | | | |
| oxononanoate | transaminase | 568 | | | |
| aminotransferase | | | | | |
| (EC 2.6.1.62) | | | | | |

Alkane Degradation - Strain 635822

| | | | | | |
|---|---|---|---|---|---|
| Coenzyme F420- | alkane | Geobacillus | 8e−69 | 34 | YP_001127577.1 |
| dependent | monoxygenase | thermodenitrificans | | | |
| N5,N10- | (ladA) | NG80-2 | | | |
| methylene | | | | | |
| tetrahydromethan | | | | | |
| opterin reductase | | | | | |
| and related flavin- | | | | | |
| dependent | oxidoreductases | | | | |
| Alkylated DNA | alkylated DNA | Serratia odorifera | 4e−106 | 90 | ZP_06639663.1 |
| repair protein | repair protein AlkB | DSM 4582 | | | |
| AlkB | (alkB) | | | | |

Alkane Degradation - Strain 322075

| | | | | | |
|---|---|---|---|---|---|
| Anaerobic nitric | Rubredoxin (rubA) | Acinetobacter sp. | 2e−9 | 46 | YP_045776.1 |
| oxide reductase | | ADP1 | | | |
| flavorubredoxin | | | | | |
| Nitric oxide | rubredoxin- | Acinetobacter sp. | 8e−53 | 34 | YP_045775.1 |
| reductase FIRd- | NAD(+) reductase | ADP1 | | | |
| NAD(+) | (rubB) | | | | |
| reductase (EC | | | | | |
| 1.18.1.—) | | | | | |
| Alkylated DNA | alkylated DNA | Serratia odorifera | 2e−56 | 53 | ZP_06639663.1 |
| repair protein | repair protein AlkB | DSM 4582 | | | |
| AlkB | (alkB) | | | | |

Naphthalene Degradation - Strain 635822

| | | | | | |
|---|---|---|---|---|---|
| Alcohol | acetaldehyde | Serratia sp. AS12 | 0 | 97 | YP_004501163.1 |
| dehydrogenase | dehydrogenase | | | | |
| (EC 1.1.1.1); | | | | | |
| Acetaldehyde | | | | | |
| dehydrogenase | | | | | |
| (EC 1.2.1.10) | | | | | |
| Alcohol | alcohol | Serratia sp. AS12 | 4e−31 | 29 | YP_004500817.1 |
| dehydrogenase | dehydrogenase | | | | |
| (EC 1.1.1.1) | GroES domain- | | | | |
| | containing protein | | | | |
| Alcohol | iron-containing | Serratia | 0 | 89 | YP_001479882.1 |
| dehydrogenase | alcohol | proteamaculans | | | |
| (EC 1.1.1.1) | dehydrogenase | 568 | | | |
| L-threonine 3- | alcohol | Serratia | 9e−31 | 28 | YP_001478632.1 |
| dehydrogenase | dehydrogenase | proteamaculans | | | |
| (EC 1.1.1.103) | | 568 | | | |
| S- | S- | Serratia | 0 | 96 | YP_001477789.1 |
| (hydroxymethyl)glutathione | (hydroxymethyl)glutathione | proteamaculans | | | |
| dehydrogenase | dehydrogenase | 568 | | | |
| (EC 1.1.1.284) | | | | | |
| Putative Rieske | ferredoxin | Pseudomonas | 2e−15 | 30 | YP_534821.1 |
| protein | component NahAb | putida | | | |
| | of naphthalene | | | | |
| | dioxygenase | | | | |
| | (nahAb) | | | | |
| NAD(P)H-flavin | reductase | Pseudomonas | 2e−21 | 29 | YP_534820.1 |
| reductase (EC | component NahAa | putida | | | |
| 1.5.1.29) (EC | of naphthalene | | | | |
| 1.16.1.3) | dioxygenase | | | | |
| | (nahAa) | | | | |
| 3- | Naphthalene 1,2- | Pseudomonas | 7e−76 | 36 | P0A110.1 |
| phenylpropionate | dioxygenase | putida | | | |
| dioxygenase, | subunit alpha, | | | | |
| alpha subunit (EC | Naphthalene 1,2- | | | | |
| 1.14.12.19) | dioxygenase IS | | | | |
| | (ndoB) | | | | |

Naphthalene Degradation - Strain 322075

| | | | | | |
|---|---|---|---|---|---|
| Alcohol | acetaldehyde | Serratia sp. AS12 | 0 | 89 | YP_004501163.1 |
| dehydrogenase | dehydrogenase | | | | |
| (EC 1.1.1.1); | | | | | |
| Acetaldehyde | | | | | |
| dehydrogenase | | | | | |
| (EC 1.2.1.10) | | | | | |
| S- | S- | Serratia sp. AS12 | 5e−157 | 73 | YP_004499970.1 |
| (hydroxymethyl)glutathione | (hydroxymethyl)glutathione | | | | |
| dehydrogenase | dehydrogenase/class | | | | |
| (EC 1.1.1.284) | III alcohol | | | | |
| | dehydrogenase | | | | |
| Ethanolamine | iron-containing | Serratia | 6e−65 | 39 | YP_001479882.1 |
| utilization protein | alcohol | proteamaculans | | | |
| EutG | dehydrogenase | 568 | | | |
| Alcohol | alcohol | Serratia | 3e−146 | 80 | YP_001478632.1 |
| dehydrogenase | dehydrogenase | proteamaculans | | | |
| (EC 1.1.1.1) | | 568 | | | |
| NAD(P)H-flavin | reductase | Pseudomonas | 5e−24 | 30 | YP_534820.1 |
| reductase (EC | component NahAa | putida | | | |
| 1.5.1.29) (EC | of naphthalene | | | | |
| 1.16.1.3) | dioxygenase | | | | |
| | (nahAa) | | | | |

1.-14. (canceled)

15. A method for reducing crude oil contamination comprising contacting a crude oil contaminated site with a crude oil degrading bacterium selected by methods comprising (i) isolating an initial crude oil degrading bacterium by culturing bacteria in a sample from the crude oil contaminated site with a medium comprising about 1% crude oil or crude oil derivatives; (ii) conditioning the initial crude oil degrading bacteria by introducing the initial crude oil degrading bacteria to the crude oil contaminated site and isolating a conditioned crude oil degrading bacterium from the crude oil contaminated site; and (iii) enhancing the conditioned crude oil degrading bacteria by culturing the conditioned bacteria one or more times with a medium comprising at least 2% crude oil or crude oil derivatives.

16. A method of proactively treating a site at risk of crude oil contamination comprising introducing a bacteria to a non-contaminated site selected by methods comprising (i) isolating an initial crude oil degrading bacterium by culturing bacteria in a sample from the non-contaminated site with a medium comprising about 0.25% crude oil or crude oil derivatives; (ii) conditioning the initial crude oil degrading bacteria by introducing the initial crude oil degrading bacteria to the non-contaminated site and isolating a conditioned crude oil degrading bacterium from the non-contaminated site; and (iii) enhancing the conditioned crude oil degrading bacteria by culturing the conditioned bacteria one or more times using a medium comprising at least 1% crude oil or crude oil derivatives.

17. The method of claim 15, wherein the crude oil composition comprises at least 15% polyaromatic hydrocarbons.

18. The method of claim 15, wherein the crude oil composition comprises at least 30% polyaromatic hydrocarbons.

19. The method of claim 15, wherein the crude oil composition comprises at least 40% polyaromatic hydrocarbons.

20. The method of claim 15, further comprising culturing the selected crude oil degrading bacterium on a culture medium comprising at least 5% crude oil or crude oil derivatives.

21. The method of claim 16, wherein the crude oil composition comprises at least 15% polyaromatic hydrocarbons.

22. The method of claim 16, wherein the crude oil composition comprises at least 30% polyaromatic hydrocarbons.

23. The method of claim 16, wherein the crude oil composition comprises at least 40% polyaromatic hydrocarbons.

24. The method of claim 16, further comprising culturing the selected crude oil degrading bacterium on a culture medium comprising at least 2% crude oil or crude oil derivatives.
